(19) United States
(12) Patent Application Publication
(10) Pub. No.: US 2023/0023462 A1
(43) Pub. Date: Jan. 26, 2023

(54) DEVICE AND KIT FOR PREVENTING TOXIC AEROSOL OF A DRUG FROM SPREADING IN THE ENVIRONMENT AND METHOD FOR THEIR USE

(71) Applicant: Biopulse S.R.L., Roma (IT)

(72) Inventors: Enrico Pierluigi Spugnini, Napoli (IT); Alfonso Baldi, Napoli (IT)

(21) Appl. No.: 17/784,184

(22) PCT Filed: Dec. 30, 2019
(86) PCT No.: PCT/IB2019/061449

§ 371 Date: Jun. 10, 2022

(30) Foreign Application Priority Data
Dec. 10, 2019 (IT) 102019000023496

(51) Int. Cl. A61J 1/20; A61J 1/14; A61M 5/32

(52) U.S. Cl. CPC A61J 1/2075; A61J 1/1412; A61M 5/3202; A61J 1/2082

(57) ABSTRACT

The invention according to the present document discloses a device 1 configured for conveying a toxic aerosol from a drug contained in a syringe (S) or in a vial (F) toward a suction pipe, comprising a cylindrical hollow element comprising an inner space configured for accommodating a syringe so that the inner walls of said space slidingly adhere to the outer walls of the syringe barrel, provided with a proximal end 2.10 which includes an opening 2.6 configured in such a way as to allow the insertion of a syringe barrel, and a flange configured for accommodating a syringe flange, and a distal end 2.11 which includes an opening 2.7 configured in such a way as to allow the cone, fitting, and needle of said syringe to pass through, and a cap configured for being connected to said cylindrical hollow element present in two configurations, one suitable for administering a drug and another suitable for taking a drug from a vial, in particular a drug potentially releasing a toxic aerosol.

The document also discloses a method for using the kit and the device according to the invention.

The present invention belongs to the field of biomedical devices for improving drug administration operations, in particular chemotherapeutic drug administration.

The present invention belongs to the technical sector of local and systemic treatment in the case of neoplastic pathologies; in particular the present invention applies to the treatment of solid metastases in human oncology and veterinary oncology.

More specifically, the present invention relates to a device and a kit for chemotherapeutic drug inoculation which conveys and draws up the toxic aerosol released by such drugs so as to limit its spreading in the environment; the present invention also discloses a method for using said device and kit for drawing up said toxic aerosol.

### PRESENT STATUS OF THE ART

Drugs potentially releasing toxic aerosols include, amongst others, chemotherapeutic drugs, which are very toxic substances having a mutagenic potential, whose administration takes place according to appropriate modes.

A variety of medical devices are known in the present status of the art, which are used to protect the operator's body from getting in contact with said toxic drugs; said devices are often devices applied onto operators' bodies in the form of masks, caps, gloves, and glasses and/or peaks; however, said devices, even though protecting operators, do not prevent aerosol from spreading in the environment. Other devices, such as extractor hoods, can only be used upon preparing drugs, i.e. while drawing up a drug in a syringe, whereas they cannot be used in the inoculation steps, other devices, such as surgical aspirators, are only used during the drug inoculation step.

Other medical devices are also known, such as Tevadaptor, applicable to the end portion of syringes in order to capture the toxic substances released upon handling said drugs; however, none of these devices allows for an administration that is completely safe for operators, patients, and the surrounding environment, especially in the case of intra-tumoral inoculation.

No devices are known in the present status of the art that minimize toxic aerosol nebulization of anti-tumoral drugs, especially in those protocols which specify an intra-tumoral injection, such as in the case of electrochemotherapy protocols.

Let's mention here below some patent documents known in the present status of the art.

Patent document CN 105617489 A discloses a device for multifunctional tumor injection. Thanks to the use of a puncture device, a controller, a pipe and a pusher, the functions of use of this multifunctional injector are enhanced, the quantity of drugs to inject is accurately measured, and injection is safe, reliable, convenient and fast to operate and makes application easier.

Patent document EP 2886146 A1 discloses a needle safety device for a drug delivery device comprising a needle cap remover suitable for capturing a needle cap by way of a positive and/or non-positive connection, wherein the cap remover comprises a top end, a shield adapted to cover a needle of the drug delivery device before and after injection, wherein the needle shield comprises a shielding end adapted to engage an injection site and at least one blocking pin which inwardly extends from the top end and/or the needle shield end into the needle cap remover and/or into the needle shield. Therefore, a need arises from the present status of the art for an innovative device that allows to convey all the toxic aerosol released by a drug, in particular a chemotherapeutic drug, toward a sucking device, while making it possible for it to be used in all steps related to handling a drug that potentially releases a toxic aerosol, such as a chemotherapeutic drug, that is to say both during the drug preparation step and during the local inoculation of said drugs, thus minimizing the risk for contaminating the environment and operators.

In particular, a need arises for a device and kit that conveys and draws up the toxic aerosol that might possibly be released from any component parts of a syringe.

### SUMMARY OF THE INVENTION

In the context of this disclosure, the terms distal and proximal are defined from the viewpoint of a person who makes an injection. Consequently, a distal direction is a direction that points toward the patient's body that receives an injection, and a distal end is an end of an element that is directed toward that patient's body. A proximal end of an element or proximal direction is directed far away from the patient's body that receives an injection and opposite to the distal end or the distal direction, respectively.

The invention discloses a device for conveying the toxic aerosol released by a drug contained in a vial or a syringe toward a suction device; said drug is generally a chemotherapeutic drug.

Further characteristics and advantages of the invention will be more apparent in the light of a detailed description of some preferred, but not exclusive, embodiments of the device and the kit used for sucking a toxic aerosol from a drug and conveying it toward a sucking device, and a method for their use, as illustrated, for explanatory non limitative purposes, with reference to the attached figures, wherein:

### BRIEF DESCRIPTION OF THE FIGURES

Other advantages, characteristics, and modes of use of the device according to the present invention will be apparent from the following detailed description of an embodiment, which is given for explanatory non-limitative purposes, and with a special reference to the attached figures, wherein:

FIG. 1—a perspective view of the distal end of a device for conveying a toxic aerosol from a drug toward a suction pipe, comprising a cylindrical hollow element 2 and a cap 3 according to a first embodiment;

FIG. 2—a perspective view of the proximal end, shown in a pre-assembling step, of a device for conveying a toxic aerosol from a drug toward a suction pipe, comprising a cylindrical hollow element 2 and a cap 3;

FIG. 3—a perspective view of the device according to the previous figures, shown in a pre-assembling step, comprising a cylindrical hollow element 2, suitable for accommodating a syringe S (shown in a pre-assembling step) and a cap 3 which joins to the cylindrical hollow element, and details of an opening 2,7 of the cylindrical hollow element placed at the distal end and a toxic aerosol delimiting space 60;

FIG. 4—a perspective view of the device according to FIG. 3 accommodating a syringe S and a cap 3 suitable for being joined to the cylindrical hollow element;

FIG. 5—a schematic view of a kit 100 comprising a device containing a syringe S connected to a suction pipe, in turn connected to a suction motor;

FIG. 6—a schematic view of a device 1 connected to a suction pipe and applied to an inoculation site, before inoculation;

FIG. 7—a schematic view of the device according to FIG. 6 upon inoculation and, consequently, in the maximum compression status of the second portion 5 of the cap 3;

FIG. 8—a perspective view of the distal end of a device for conveying a toxic aerosol from a drug toward a suction pipe, comprising the cylindrical hollow element 2 and the cap 3′ according to a second embodiment, assembled and consequently containing a vial F;

FIG. 9—a perspective view of a device for conveying a toxic aerosol from a drug toward a suction pipe, shown in a pre-assembling step, comprising the cylindrical hollow element 2 and the cap 3′,

FIG. 10—a schematic view of a kit 100 comprising a device containing a syringe S, formed of the cylindrical hollow element 2 and the cap 3′, containing a vial F, connected to a suction pipe, in turn connected to a suction motor.

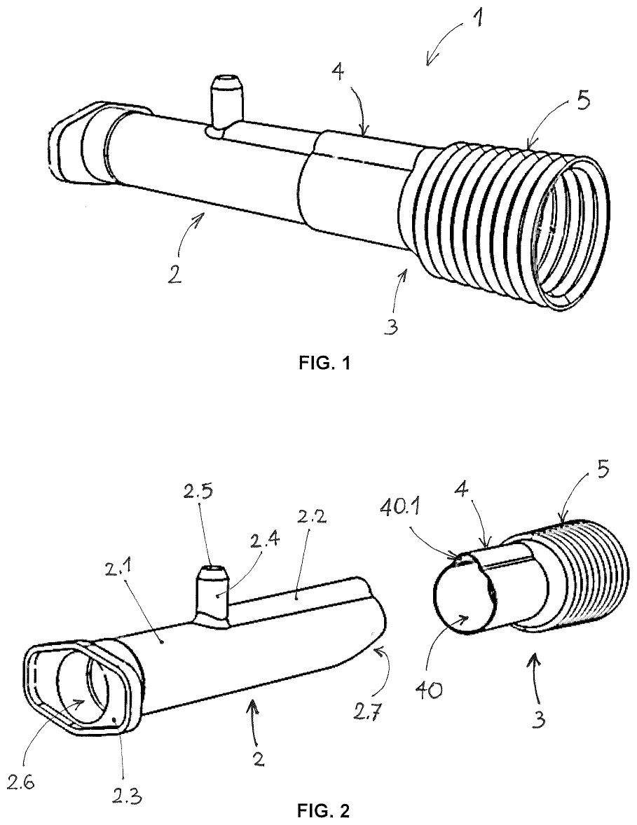

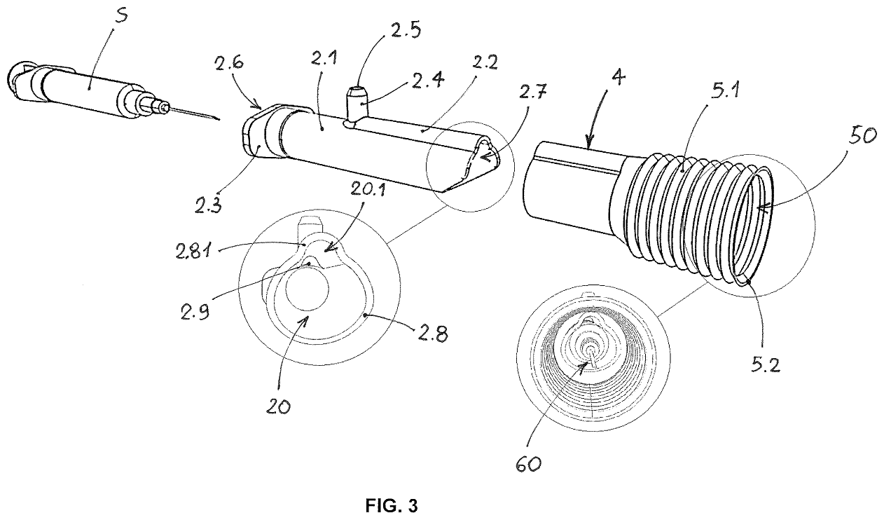

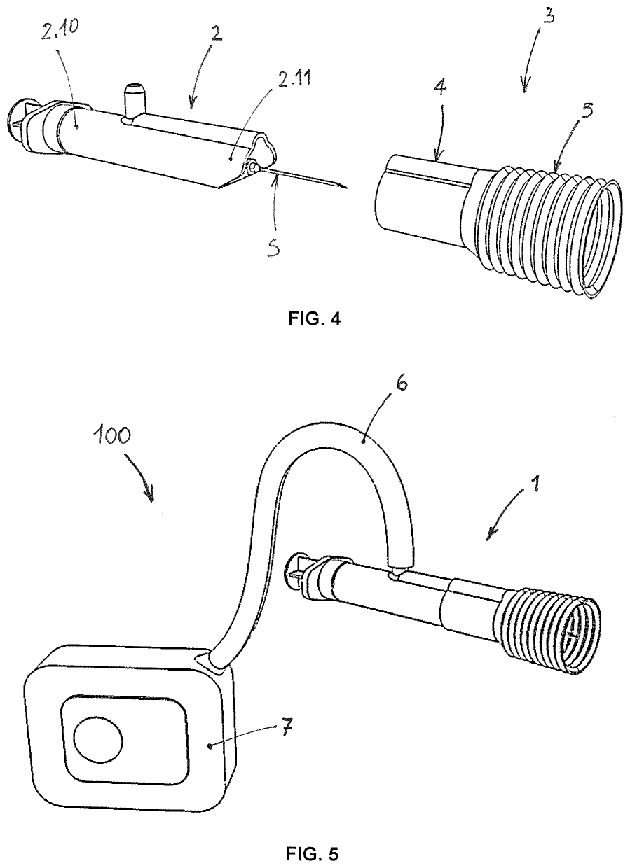

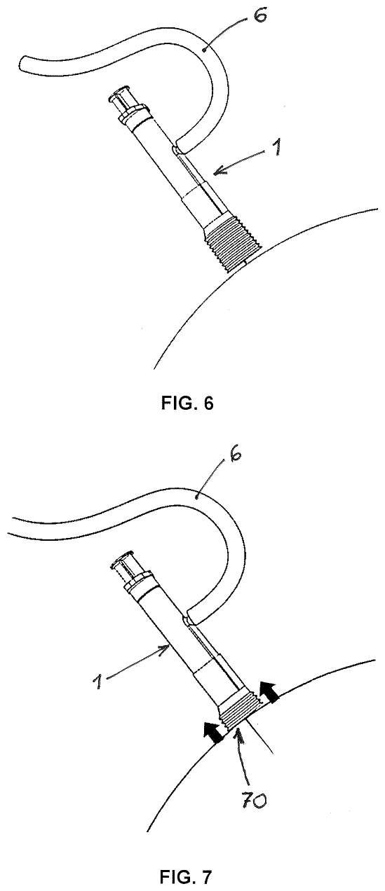

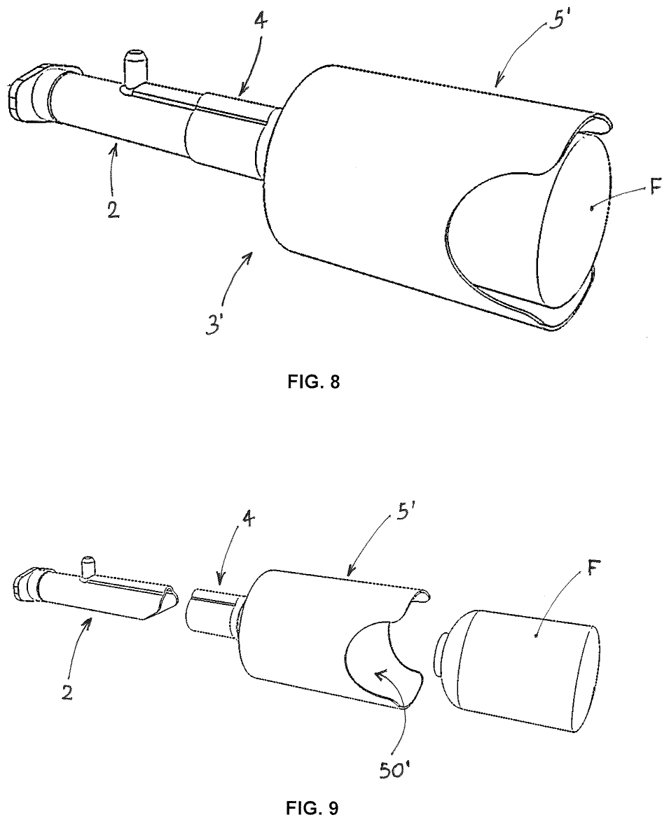

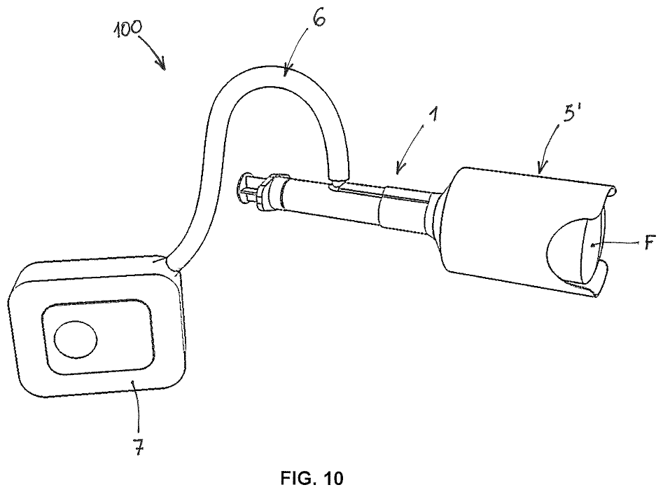

### LEGEND

1. device for conveying the toxic aerosol from a drug toward a suction pipe;
2. cylindrical hollow element;
   2.1. outer surface of the first cylindrical hollow element;
   2.2 convexity present on the outer surface 2.1 of the first cylindrical hollow element 2;
   2.3. flange of the first cylindrical hollow element;
   2.4. fitting for the opening 2.5;
   2.5. orifice placed on the outer surface 2.1 of the first cylindrical hollow element 2;
   2.6 opening of the cylindrical hollow element 2 placed at the proximal end 2.10;
   2.7 opening of the cylindrical hollow element placed at the distal end 2.11;
   2.8. distal edge of the cylindrical hollow element 2;
      2.81. off-the-edge present on the edge 2.8 of the end 2.11;
   2.9. orifice present at the end 20′ of an internal groove of the cylindrical hollow element;
   2.10. proximal end of the cylindrical hollow element;
   2.11. distal end of the cylindrical hollow element;
3. cap according to a first configuration;
3′. cap according to a second configuration;
4. first hollow portion of the cap 3; 3′;
5. second hollow portion of the cap 3 according to a first configuration;
   5.1 outer surface folded onto itself on the second portion 5 of the cap 3;
   5.2 lower edge of the second cylindrical hollow portion 5 of the cap 3;
   5.3. distal end of the second hollow portion 5, 5′;
5′. second hollow portion of the cap 3′ according to a second configuration;
6. suction pipe
7. suction motor;
8. suction motor filters;
20. inner space of the cylindrical hollow element 2;
   20.1 inner longitudinal groove of the cylindrical hollow element;
40. inner space of the portion 4;
   40.1. inner groove of the space 40 configured for accommodating a convexity 2.2;
50. inner space of the second portion 5;
50′. inner space of the second portion 5′ configured for accommodating a drug-containing vial;
60. toxic aerosol delimiting space, comprising the inner space 20 and the inner spaces 20, 40, 50, and 50′ communicating with each other;
70. closed space delimited by the cap 3 whenever it adheres to a surface;
100. kit for conveying and sucking up a toxic aerosol from a drug.

### DETAILED DESCRIPTION OF THE INVENTION

The device 1 according to this invention is a device and kit 100 for inoculation of drugs potentially releasing a toxic aerosol in the environment, such as chemotherapeutic drugs; the device according to this invention is a medical device configured for accommodating a syringe and for limiting the spreading of toxic aerosol in the environment while handling a toxic drug, i.e. both in the inoculation step and in the drug preparation step before inoculation, such as, for instance, drawing up a drug from a vial.

In particular, the device according to this invention is a device for conveying, toward a suction pipe, a toxic aerosol released and delimited by the device itself.

The device according to this invention comprises two embodiments having a cylindrical hollow element 2, which represents a sheath for containing a syringe, in common; the cap used for covering the needle features two different configurations, one of which is compressible and consequently usable for inoculating a toxic drug contained in a syringe (S), and another is used for taking a drug from a vial (F).

Said device 1 for conveying a toxic aerosol from a drug contained in a syringe (S) or in a vial (F) toward a suction pipe comprises a cylindrical hollow element comprising an inner space configured for accommodating a syringe, so that the inner walls of said space slidingly adhere to the outer walls of the syringe barrel, provided with a proximal end 2.10 which includes an opening 2.6 configured in such a way as to allow for the insertion of a syringe barrel, and a flange configured for accommodating a syringe flange, and a distal end 2.11 which includes an opening 2.7 configured in such a way as to allow the cone, fitting, and needle of said syringe to pass through, and a cap configured for being connected to said cylindrical hollow element.

Said device 1 is characterized in that it comprises:
said cylindrical hollow element 2 which, at the distal end, is provided with said opening 2.7 featuring a circular cross section cut to 45°, such as to allow to vary the tilt of the device 1 with respect to the longitudinal axis crossing said cylindrical hollow element 2, said opening 2.7 being provided with an off-the-edge 2.81 featuring an almost semi-circular cross section on the edge 2.8, in a position opposite to that of the 45° cutting, said cylindrical hollow element 2 being provided, on its outer surface 2.1 and along its longitudinal axis, with a convexity 2.2 integrally connected thereto, which a longitudinal groove 20.1 of the inner space 20 terminating in said off-the-edge 2.81 on the edge 2.8 of the distal end corresponds to, said cylindrical hollow element 2 being connected to said cap 3; 3′ at said distal end 2.11 in a removable manner;
said longitudinal groove 20.1 includes, at a proximal end, an orifice 2.9 which is connected to the orifice 2.5 present on the outer surface 2.1 and is configured to form a duct suitable for conveying, together with the outer wall of a syringe barrel an aerosol toward the orifice 2.9;
said cap 3; 3′ being provided with a first hollow portion 4 having a shape conjugated to that of the end 2.11 of said first cylindrical hollow element 2, so as to accommodate said cylindrical hollow element 2 internally thereto, a groove 40.1 being present in the inner space 40 of said first portion 4, configured for accommodating the convexity 2.2 of the cylindrical hollow element 2, and a second hollow portion 5, 5′ integrally connected to said first portion 4 delimiting an inner space 50, 50′ open at the distal end 5.3, suitable for delimiting, together with the inner space 40 of said first portion 4 and together with the inner space 20 of said cylindrical hollow element 2, being communicating with each other, a unique space 60 delimiting the toxic aerosol and conveying it by way of the grooves 20.1 and 40.1 toward the orifice 2.9, so as to allow said toxic aerosol to flow through toward the outer orifice 2.5 connectable to a suction pipe.

Said device is thus configured in such a way as to convey all the toxic aerosol that might be released from any component parts of a syringe toward a suction device, i.e. both in the case of any leakages of micro-drips of drug or its aerosol, if any, from a syringe needle tip, and in the case of a drug or its aerosol getting out from its connection to the cone, or both in the case of a drug or its aerosol getting out from the inoculation point immediately after inoculation, and a drug or its aerosol getting out from a vial immediately after taking the drug.

In a first configuration of the invention, said cap 3 includes a second portion 5 whose outer surface 5.1 is folded on itself like an accordion, so as to be expandable and compressible; in this configuration, said second portion 5 is made from a soft material, preferably a silicone material, so as to foster the movement of the device upon inoculation; more preferably, said silicone material will be transparent so as to allow for an operator to clearly see the inoculation point and adjust the position of a syringe needle accordingly.

Said cap 3 is configured so that, upon inoculation, it adheres with its edge 5.2 to the surface to be treated thus delimiting a closed space 70; this allows to prevent toxic aerosol from getting out.

The silicone protection 5 makes thus it possible to form a closed area 70 around the inoculation site, which allows, first of all, to prevent a harmful aerosol released by a drug, generally an antitumoral drug, from being spread in the environment; said aerosol will subsequently be conveyed, by way of the grooves 20.1 and 40.1, toward the opening 2.9 in order for it to be conveyed toward a suction pipe.

Said cap 3 is also configured so that subsequently, while inoculation is in progress, varies its own configuration, in that it passes from an expansion status wherein a syringe needle accommodated inside said cylindrical hollow element 2 is internal to said space 70, to a compression status wherein the syringe needle gets out beyond the edge 5.2 of said cap 3, following the movements of the syringe, hence of the cylindrical hollow element 2, toward the patient's body upon drug inoculation.

The silicone protection described with reference to the second portion 5 makes thus it possible to form a closed area, around the inoculation site, internally to which the released toxic aerosol is limited and conveyed toward a suction pipe 6 by way of the grooves 40.1 and 20.1 and through the orifice 2.9.

The device according to this invention is also available in a second configuration suitable for limiting the spreading of a toxic aerosol from a drug contained in a vial (F) in the environment and conveying it toward a suction device; in this configuration, said cap 3′ includes a second portion 5′ having a diameter greater than that of the first portion 4 and including an inner space 50′ configured for accommodating a drug-containing vial; in this embodiment, said second portion 5′ is made from a rigid plastic material or from a semi-rigid silicone material; in this case too, a transparent material is preferred in order to be able to inspect the vial being handled.

After inserting a vial inside the inner space 50′, a gap will be formed all around the vial, from which any leakages of drug taking place while taking it will be conveyed and subsequently drawn up.

The device according to this invention is configured so that, in both embodiments, the first portion 4 of said cap 3; 3′ is slidingly connected to said cylindrical hollow element 2 so as to accommodate the end 2″ of said cylindrical hollow element 2 in the inner space 40, along three quarters of its length, so as to reciprocally secure them.

Also, at the proximal end the convexity 2.2 ends, in an opening 2.5 which protrudes toward the outside of said cylindrical hollow element 2 by way of a fitting 2.4, said opening 2.5 being configured for being connected to a pipe where the toxic aerosol will be conveyed.

Said opening 2.5 is positioned below the flange 2.3.

Also, said flange 2.3 is configured so as to accommodate and restrain the flange of a syringe barrel.

The invention also discloses a kit 100 for conveying and drawing up a toxic aerosol from a drug, comprising a device 1 consisting of at least one cylindrical hollow element 2, at least one cap 3 and at least one cap 3′, at least one suction pipe 6 configured for connecting the opening 2.5 to at least one suction motor 7, and a suction motor 7 configured for drawing up the air contaminated by a toxic aerosol from a drug and purifying it; said suction motor 7 is provided with filters 8 for absorbing the toxic aerosol from a drug so as to purify the output air.

The suction motor will exert a suction force that will allow all the aerosol conveyed in the one space 60 inside the suction pipe 6 to flow through, so as to totally prevent any leakages of toxic substances during inoculation.

The air drawn up by the motor will subsequently be purified through specific removable filters, which will capture molecules present in the aerosol, so as to output clean air; said filters are also chemotherapeutic substance-specific filters.

The filters used for purifying air from the chemical substances present are active charcoal filters.

Said active charcoal filters are filters intended for protection from solid and liquid aerosols, with or without oil, and comprise:
an electrostatic filtering means for particulate retention;
an anti-odor filtering element for protection from ozone;
a filtering element made from a polypropylene and polyester woven-non-woven fabric.

The kit according to this invention is intended for using chemotherapeutic drugs.

The invention also provides a method for using the device and the kit according to the invention comprising the following steps:
a) assembling the cap 3′ on the cylindrical hollow element 2;
b) inserting a sterilized syringe internally to the cylindrical hollow element 2;
c) locking the flange of the syringe barrel in the flange 2.3 of the cylindrical hollow element 2;
d) turning on the suction motor 7 connected to the device 1, by way of the suction pipe 6;
e) inserting a vial (F) containing the drug to be taken in the syringe (5);
f) taking advantage of draught to draw up the drug in the sterilized syringe toward the outside of the syringe plunger;
g) closing the needle with an appropriate plug, wherein said step whereby the drug is taken from a vial takes place simultaneously with a step whereby the toxic aerosol released by the drug is drawn up by way of a suction motor.

The method according to the invention jointly comprises the following further steps:
h) disassembling the cap 3′;
i) assembling the cap 3;
j) making the cap 3 adhere to the surface to be treated so as to form a closed space 70;
k) making needle pierce the inoculation site, whereby the second hollow portion 5 of the cap 3 changes its configuration so as to allow the needle to completely enter the surface to be treated;
l) pushing the syringe plunger to expel the drug from the needle;
m) making the needle get out from the inoculation site and closing the needle with an appropriate plug;
n) turning the suction motor off,
   wherein the steps whereby a syringe needle enters and gets out from the inoculation site respectively and the step whereby the drug gets out from a syringe needle take place simultaneously with a step whereby the toxic aerosol released by the drug is drawn up.

In one embodiment according to the invention, namely whenever pre-filled syringes are used containing toxic drugs, such as some chemotherapeutic drugs, steps a) thru i) are replaced by the following steps:
mounting the cap 3;
turning on the suction motor 7 connected to the device 1 via the suction pipe 6;
inserting a pre-filled syringe, containing a drug potentially releasing a toxic aerosol, in the inner space of the cylindrical hollow element 2 of the device 1;
locking the flange of a syringe barrel in the flange 6 of the cylindrical hollow element 2.

In the method according to this invention, the step of drawing up toxic aerosol from a drug is followed by a further step, whereby the air output from the suction motor 7 is purified by way of filters 8 of the suction motor 7.

The method according to the invention makes it possible to convey an aerosol potentially released by a syringe in every step of its use, by first assembling the cap 3′, intended for use with a vial, on the cylindrical element, then the cap 3 intended for drug inoculation; the method according to the invention is particularly used to inoculate chemotherapeutic drugs.

The invention also describes the use of the device 1 and of the kit 100 for handling a drug, contained in syringes or vials, that potentially releases a toxic aerosol in the environment.

The device and kit according to the invention will be used in the case of use of drugs such as antitumoral drugs and drugs for genic therapies.

These drugs include, for explanatory purposes only, some chemotherapeutic drugs for intra-lesional application: platinum-based chemotherapeutic drugs (cisplatinum, carboplatinum, and the like), bleomycin, mitomicyn, fluorouracil, and infusional chemotherapeutic drugs such as vincristine, cyclophosphamide, doxorubicin, cytarabine, actinomycin, mechlorethamine, vinblastine, vindesine, and other chemotherapeutic infusional drugs.

The device and kit according to the invention aims at significantly reducing the risk of environmental contamination deriving from an increasing use of drugs potentially releasing toxic aerosols, such as chemotherapeutic drugs; as a matter of fact, the device and kit according to the invention aim at conveying and drawing up all the aerosol that might be released from any individual parts of a syringe and in every toxic drug handling step.

The device and kit according to the present invention also aims at reducing contamination in health operators in charge of providing assistance during chemotherapeutic drug taking and inoculation steps.

Said device and kit can be used both for human and for veterinary applications.

The scope of the invention is susceptible of numerous modifications and variants, all falling within the inventive concept set forth in the attached claims.

All details can be replaced by other technically equivalent elements, according to the requirements, without departing from the scope of protection of the present invention. Even though the scope has been described with a special reference to the attached figures, the reference numerals used in the description and in the claims are used for making the understanding of the invention easier and do not constitute any limitations to the claimed scope of protection.

1. A device for conveying a toxic aerosol from a drug contained in a syringe or in a vial toward a suction pipe, comprising:
a cylindrical hollow element comprising an inner space configured for accommodating a syringe, so that the inner walls of said inner space slidingly adhere to the outer walls of a syringe barrel, the cylindrical hollow element provided with a proximal end which includes an opening configured in such a way as to allow the insertion of the syringe barrel, and a flange configured for accommodating a syringe flange, and a distal end which includes an opening configured in such a way as to allow a cone, fitting, and needle of said syringe to pass through; and
a cap configured for being connected to said cylindrical hollow element, wherein:
said cylindrical hollow element is provided, at its distal end, with said opening, featuring a circular cross section cut to 45° suitable for making it possible to vary a tilt of the device with respect to a longitudinal axis passing through said cylindrical hollow element, said opening being provided with an off-the-edge having an almost semi-circular cross-section in an edge, in a position opposed to the 45° cutting, said cylindrical hollow element being provided, on an outer surface and along the longitudinal axis, with a convexity integrally connected thereto, which a longitudinal groove of the inner space terminating in said off-the-edge on the edge of the distal end corresponds to, said cylindrical hollow element being connected to said cap at said distal end in a removable manner;
said longitudinal groove includes, at its proximal end, an end orifice which is connected to an outer orifice present on the outer surface and is configured to form, together with the outer wall of the syringe barrel, a duct suitable for conveying an aerosol toward the end orifice;
said cap is provided with a first hollow portion having a shape conjugated to that of the end of said cylindrical hollow element, so as to accommodate said cylindrical hollow element internally thereto, a groove being present in an inner space of said first portion, configured for accommodating the convexity of the cylindrical hollow element, and a second hollow portion integrally connected to said first portion delimiting an inner space of the second portion open at the distal end suitable for delimiting, together with the inner space of said first portion and together with the inner space of said cylindrical hollow element, communicating to each other, a unique space for delimiting the toxic aerosol and conveying it toward the end orifice by way of the grooves, so as to allow for said toxic aerosol to flow through toward the outer orifice connectable to a suction pipe.

2. The device according to claim 1, wherein said cap includes a second portion whose outer surface is folded onto itself like an accordion, so as to be expandable and compressible.

3. The device according to claim 2, wherein said second portion is made from a soft material, preferably a silicone material.

4. The device according to claim 1, wherein said cap is configured in such a way as to adhere to a surface with its edge thus delimiting a closed space.

5. The device according to claim 1, wherein said cap is configured in such a way as to vary its own configuration and to switch from an expansion status wherein the needle of a syringe accommodated inside said cylindrical hollow element is internal to said inner space thereof, to a compression status wherein the syringe needle gets out beyond an edge of said cap.

6. The device according to claim 1, wherein said cap includes the second portion having a diameter greater than that of the first portion and being provided with the inner space of the second portion configured for accommodating a vial containing a drug.

7. The device according to claim 6, wherein said second portion is made from a rigid or semi-rigid plastic material.

8. The device according to claim 1, wherein said first portion of said cap is slidingly connected to said cylindrical hollow element so as to accommodate the end of said cylindrical hollow element in the inner space of the first portion along three quarters of its length so as to allow to reciprocally secure them.

9. The device according to claim 1, wherein the outer orifice protrudes toward the outside of said cylindrical hollow element by way of a fitting, said opening outer orifice being configured for being connected to a suction pipe.

10. The device according to claim 1, wherein the outer orifice is positioned below the flange.

11. The device according to claim 1, wherein said flange is configured so as to accommodate and restrain the flange of a syringe barrel so as to lock it internally thereto.

12. A kit for conveying and drawing up a toxic aerosol from a drug comprising the device according to claim 1, wherein the kit comprises the device consisting of:
at least one cylindrical element,
at least two caps;
at least one suction pipe configured for connecting the outer orifice to a suction motor;
a suction motor configured for drawing up the contaminated air from a toxic aerosol of a drug and purifying the contaminated air.

13. The kit for drawing up a toxic aerosol from a drug comprising the device according to claim 12 wherein said suction motor is provided with filters for absorbing the toxic aerosol from a drug.

14. A method for using the device according to claim 1 comprising the following steps:
a) assembling the cap on the cylindrical hollow element;
b) inserting a sterilized syringe into the cylindrical hollow element;
c) blocking the flange of a syringe barrel in the flange of the cylindrical hollow element;
d) turning on a suction motor connected to the device through a suction pipe;
e) inserting a vial containing the drug to be drawn up into the syringe;
f) taking advantage of draught to draw up the drug in the sterilized syringe to the outside of a syringe plunger;
g) closing the needle with its appropriate plug, wherein said step whereby a drug is taken from a vial takes place simultaneously with a step whereby the toxic aerosol released by the drug is drawn up.

15. The method for using the device according to claim 14, jointly comprising the following further steps:
h) disassembling the cap for use with the vial;
i) assembling the cap for use with an inoculation;
j) making the second portion adhere to a surface to be treated so as to form a closed space;
k) inserting the needle into an inoculation site wherein the second cylindrical hollow portion varies its own configuration so as to allow for the needle to completely enter the surface to be treated;
l) pushing the syringe plunger to make the drug get out from the needle;
m) taking the needle out of the inoculation site and closing the needle with its appropriate plug;
n) turning the suction motor off; wherein said step whereby the drug gets out from syringe needle takes place simultaneously with a step whereby the toxic aerosol released by the drug is drawn up.

16. The method for using the device according to claim 15, wherein steps a) thru i) are replaced by the following steps:
assembling the cap;
turning on the suction motor connected to the device through the suction pipe;
inserting a pre-filled syringe, containing a drug potentially releasing a toxic aerosol, in the inner space of the cylindrical hollow element of the device;
locking the flange of a syringe barrel in the flange of the cylindrical hollow element.

17. The method according to claim 16 wherein, after drawing up a toxic aerosol from a drug, there is a further step whereby the air output from the suction motor is purified by way of filters of the suction motor.

18. (canceled)

19. The device according to claim 1, wherein said second portion is made from a rigid or semi-rigid plastic material.
